(19) United States
(12) Patent Application Publication
(10) Pub. No.: US 2019/0159780 A1
(43) Pub. Date: May 30, 2019

(54) METHOD OF PERFORMING AN OSTOMY PROCEDURE

(71) Applicant: Covidien LP, Mansfield, MA (US)

(72) Inventor: AVVLN Srinivasa Murthy Aravalli, Tanuku (IN)

(21) Appl. No.: 16/202,505

(22) Filed: Nov. 28, 2018

Related U.S. Application Data
(60) Provisional application No. 62/591,710, filed on Nov. 28, 2017.

(51) Int. Cl. A61B 17/11; A61B 17/115; A61B 17/34

(52) U.S. Cl. CPC A61B 17/1114; A61F 5/445; A61B 17/3415; A61B 17/1155

(57) ABSTRACT

A method of performing an ostomy procedure includes, inter alia, forming a first incision in an abdominal wall of a patient; inserting an anvil head assembly of an anvil assembly through the first incision with the anvil head assembly in a tilted position in relation to a center rod assembly of the anvil assembly; moving the anvil head assembly from the tilted position to an operative position; coupling the anvil assembly to an anvil retainer of the circular stapling device; approximating the anvil head assembly in relation to a staple cartridge of the shell assembly to clamp the rectus sheath of the abdominal wall between the anvil head assembly and the staple cartridge; and firing the circular stapling device.

### CROSS-REFERENCE TO RELATED APPLICATIONS

This application claims the benefit of and priority to U.S. Provisional Patent Application No. 62/591,710 filed Nov. 27, 2017, the entire disclosure of which is incorporated by reference herein.

### BACKGROUND

### 1. Technical Description

The present disclosure is directed to a method of performing an ostomy procedure and, more particularly, to a method of performing an ostomy procedure to avoid complications such as parastomal herniation.

### 2. Background of Related Art

Exteriorization of an internal body vessel such as the intestine is called a stoma. Stomas may be created in conjunction with an ostomy procedure, e.g., colostomy or ileostomy, by suturing a bisected portion of an intestine to the abdominal wall to provide internal access into the intestine for collecting fecal matter. Parastomal herniation is the most significant and frequent complication associated with patients that have had ostomy procedures. Parastomal herniation generally refers to a bulge or swelling around the stoma that leads to problems with stoma function.

Parastomal herniation may result when the abdominal muscles, e.g., the rectus sheath, separate from the stoma. This separation, which usually occurs gradually and may worsen over time, may allow an internal body organ, generally a section of the bowel, to enter the space between the abdominal muscles and the stoma, which may result in strangulation of the internal body organ.

A continuing need exists in the art for an improved procedure for forming a stoma to minimize the likelihood of parastomal herniation from occurring.

### SUMMARY

One aspect of the disclosure is directed to a method of performing an ostomy procedure including forming a first incision in an abdominal wall of a patient; inserting an anvil head assembly of an anvil assembly through the first incision with the anvil head assembly in a tilted position in relation to a center rod assembly of the anvil assembly; positioning the anvil head assembly in the abdominal cavity of the patient; moving the anvil head assembly from the tilted positon to an operative position; coupling the anvil assembly to an anvil retainer of the circular stapling device; inserting a shell assembly of the circular stapling device through the incision; approximating the anvil head assembly in relation to a staple cartridge of the shell assembly to clamp the rectus sheath of the abdominal wall between the anvil head assembly and the staple cartridge; and firing the circular stapling device to form a circular opening in the rectus sheath that is reinforced by an annular array of staples.

In embodiments, the method further includes removing the anvil assembly from the abdominal cavity.

In embodiments, removing the anvil assembly from the abdominal cavity is conducted through the first incision.

In certain embodiments, the method includes moving the anvil head assembly to the tilted position prior to removing the anvil head assembly from the abdominal cavity.

In some embodiments, the method includes separating the anvil assembly from the anvil retainer before removing the anvil assembly from the abdominal cavity.

In embodiments, the method includes securing a portion of a bowel of a patient to the abdominal wall to form a stoma.

In certain embodiments, securing a portion of the bowel to the abdominal wall includes securing the portion of the bowel to a dermis of the abdominal wall of the patient.

In some embodiments, securing the portion of the bowel of the patient to the abdominal wall is performed with the circular stapling device.

In embodiments, securing the portion of the bowel of the patient to the abdominal wall includes inserting the anvil head assembly of the circular stapling device through the first incision and clamping the portion of the bowel to the outer cutaneous layer of the abdominal wall to form a stoma.

In certain embodiments, the method includes forming a circular opening in the outer cutaneous layer of the abdominal wall.

In some embodiments, the method includes removing the anvil assembly from the stoma.

In embodiments, the method includes measuring a diameter of a section of bowel to determine an appropriate size for the circular stapling device.

### BRIEF DESCRIPTION OF THE DRAWINGS

Various embodiments of the presently disclosed method of performing an ostomy procedure are described herein below with reference to the drawings, wherein:

FIG. 1 is a side perspective view of a circular stapling device for use in preforming the presently disclosed methods of performing an ostomy procedure;

FIG. 2 is an exploded side perspective view of a distal end portion of the circular stapling device shown in FIG. 1;

FIG. 3 is a side perspective view of an organ measuring device positioned through a cannula that extends through an incision in an abdominal wall of a patient with a distal portion of the organ measuring device positioned about an organ to be exteriorized;

FIG. 4 is a side view of an anvil assembly of the circular stapling device shown in FIG. 1 positioned adjacent to an incision formed in the abdominal wall of the patient with an anvil head of the anvil assembly in a tilted position;

FIG. 5 is a side view of the anvil assembly of the circular stapling device shown in FIG. 1 extending through the incision formed in the abdominal wall of the patient with the anvil head of the anvil assembly in a tilted position;

FIG. 6 is a side view of a distal portion of the circular stapling device shown in FIG. 1 with the anvil assembly extending through the incision formed in the abdominal wall of the patient and the anvil head of the anvil assembly in an operative position prior to attachment of the anvil assembly to an anvil retainer of the circular stapling device;

FIG. 7 is a side view of the distal portion of the circular stapling device shown in FIG. 6 with the anvil assembly extending through the incision formed in the abdominal wall of the patient and the anvil head of the anvil assembly in the operative position with the anvil assembly attached to an anvil retainer of the circular stapling device and the anvil assembly in a unclamped position;

FIG. 8 is a side view of the distal portion of the circular stapling device shown in FIG. 6 with the anvil assembly extending through the incision formed in the abdominal wall of the patient and the anvil head of the anvil assembly in the operative position with the anvil assembly attached to an anvil retainer of the circular stapling device and the anvil assembly in a clamped position;

FIG. 8A is a cross-sectional view taken along section line 8A-8A of FIG. 8;

FIG. 9 is a side view of the anvil assembly shown in FIG. 8A separated from the anvil retainer of the circular stapling device with the anvil head of the anvil assembly in a tilted position as the anvil assembly is removed from the patient;

FIG. 9A is a side view of the distal portion of the circular stapling device with a fresh shell assembly as the circular stapling device is fired to secure a section of bowel to the dermis of the abdominal wall;

FIG. 10 is a top view of a stoma stapled to the dermis of the abdominal wall; and

FIG. 11 is a side perspective view of the stoma and the abdominal wall illustrating the section of bowel in phantom.

### DETAILED DESCRIPTION OF EMBODIMENTS

The presently disclosed methods of performing an ostomy procedure will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel.

During an ostomy procedure, e.g., colostomy or ileostomy, a surgically created opening is formed in the abdominal wall for the discharge of body waste. Generally, a portion of the bowel, e.g., the colon or intestine, is secured to the abdominal wall to deliver body waste to the surgically created opening. The portion of the bowel that is visible protruding through the surgically created opening is called a stoma. The most significant and frequent complication associated with patient's that have had ostomy procedures is parastomal herniation. Parastomal herniation generally refers to a bulge or swelling around the stoma that leads to problems with stoma function. A parastomal hernia can occur when edges of the stoma separate from abdominal muscles such that abdominal contents, generally a portion of the bowel, bulge through the space between the abdominal muscles and the stoma. The presently disclosed method of performing an ostomy procedure provides a reinforced opening through tissue defining the abdominal wall to minimize the likelihood of parastomal herniation.

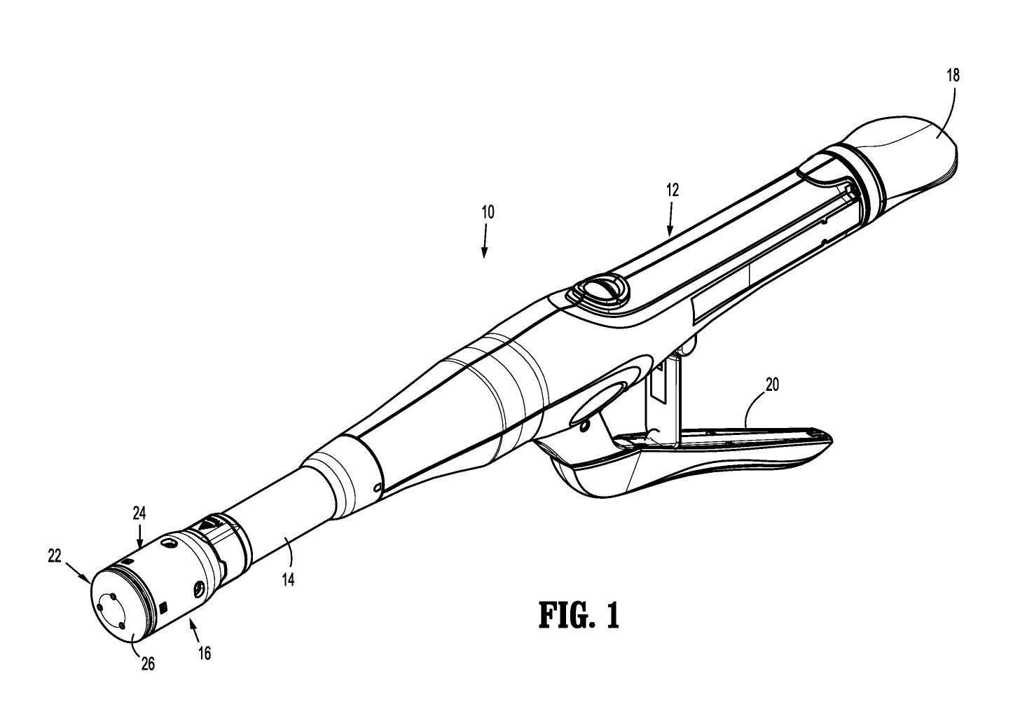

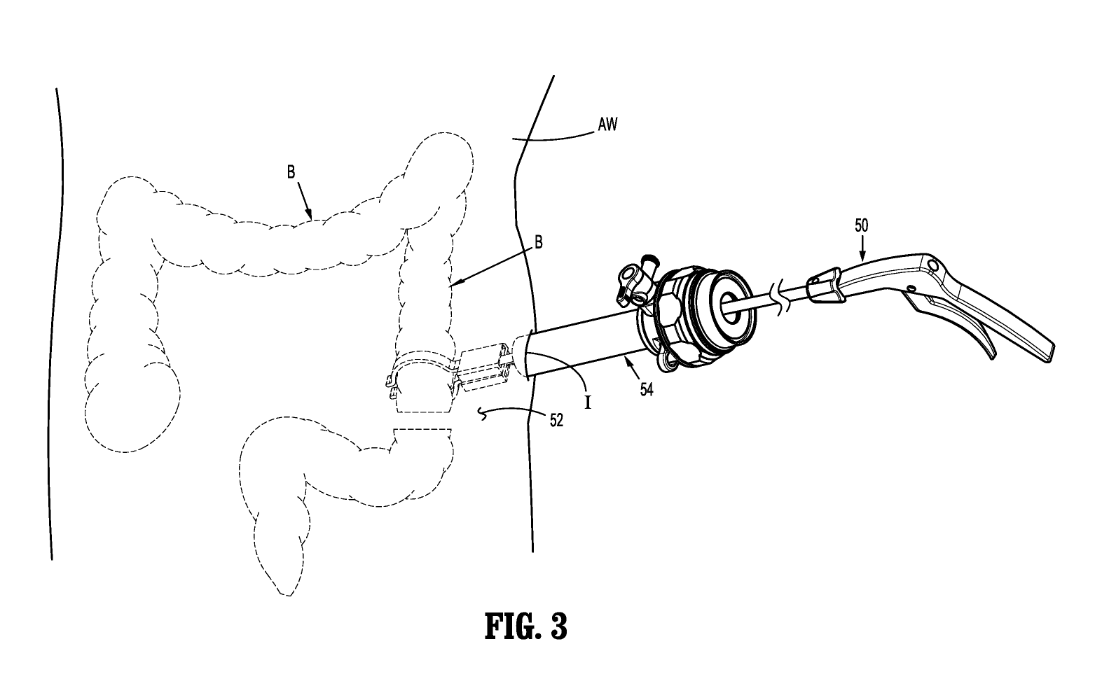

Referring to FIG. 1, in the presently disclosed method of performing an ostomy procedure, a circular stapling device 10 is provided to secure an internal organ such as a portion of the bowel “B” (FIG. 3) to the abdominal wall “AW” (FIG. 3). In embodiments, the circular stapling device 10 includes a handle assembly 12, a body 14 extending from the handle assembly 12, and a tool assembly 16 supported on a distal portion of the body 14. The handle assembly 12 includes a manually operated rotation knob 18 for approximating the tool assembly 16 as described in detail below and a manually operated firing trigger 20 for firing the tool assembly 16. For a more detailed description of the circular stapling device 10, see U.S. Pat. No. 9,307,994 (“'994 Patent”) which is incorporated herein by reference in its entirety.

Although the circular stapling device 10 is illustrated as being manually operated, it is envisioned that the stapling device 10 may also be electrically powered. For a detailed description of the structure and function of an exemplary electromechanical device, see U.S. patent application Ser. No. 13/484,975, filed on May 31, 2012, now published as U.S. Patent Publication No. 2012/0253329 which is incorporated herein by reference in its entirety.

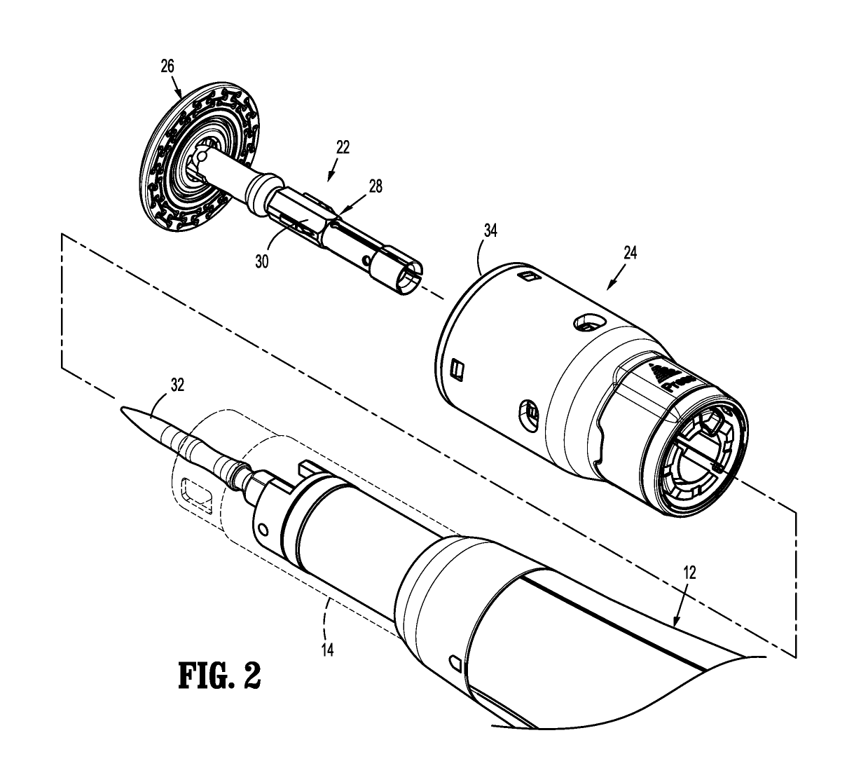

Referring also to FIG. 2, the tool assembly 16 includes an anvil assembly 22 and a shell assembly 24. The anvil assembly 22 includes an anvil head assembly 26 and an anvil center rod assembly 28. The center rod assembly 28 includes a center rod 30 that is adapted to releasably engage an anvil retainer 32 of the circular stapling device 10. In embodiments, the anvil head assembly 26 is pivotably supported on a distal portion of the center rod assembly 28 and is movable between an operative position and a tilted position as described in further detail below. For a more detailed description of known anvil assemblies with tiltable anvil head assemblies, see U.S. Pat. Nos. 9,532,781 and 6,957,758, and the '994 Patent which are incorporated herein in their entirety by reference.

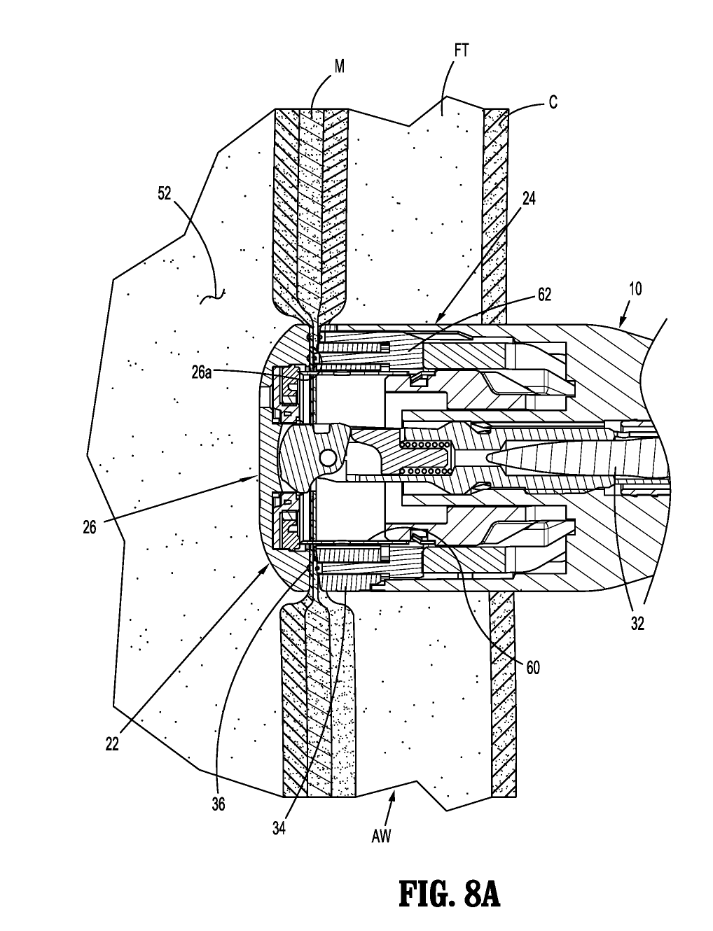

The shell assembly 24 includes a staple cartridge 34 that supports an annular array of staples 36 (FIG. 8A). In embodiments, the shell assembly 24 is adapted to be releasably coupled to the distal portion of the body 14 to allow for replacement of the shell assembly 24 to facilitate reuse of the circular stapling device 10 after the staples 36 have been fired from the staple cartridge 34. For a more detailed description of a circular stapling device having a shell assembly 24 that is releasably coupled to the body of the stapling device, see U.S. application Ser. No. 14/859,590 (“the '590 application”) which is incorporated herein by reference in its entirety.

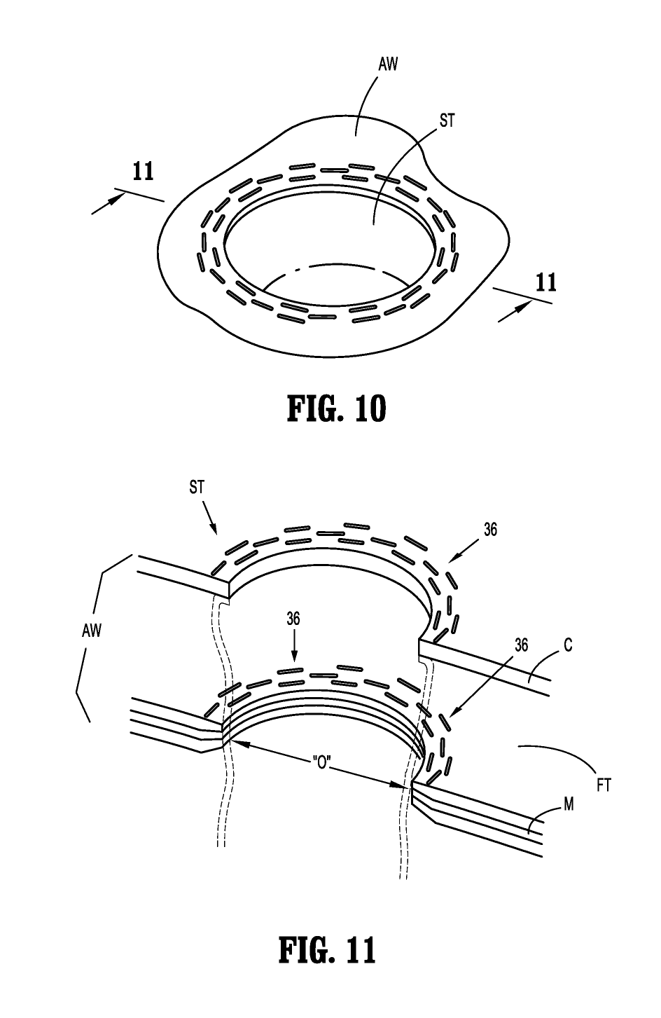

Referring to FIG. 3, in order to select the appropriate size circular stapling device 10 to perform the presently disclosed ostomy procedure, the size or diameter of the bowel “B” is measured to determine the appropriate diameter of the hole that is to be formed through the abdominal wall “AW”. The size of the hole “O” (FIG. 11) should be selected to correspond to the outer diameter of the bowel “B”. In embodiments, the diameter of the bowel “B” can be measured using a bowel measuring instrument 50. The bowel measuring instrument 50 can be inserted into the abdominal cavity 52 through a cannula 54 to access a portion of the bowel “B” to be exteriorized to form a stoma “ST” (FIG. 10) and measure its diameter. The cannula 54 can be inserted through an incision “I” formed through the abdominal wall “AW”. As used herein, the abdominal wall “AW” includes an outer cutaneous layer “c” (e.g., epidermis, dermis, and hypodermis), a layer of fatty tissue “FT”, and an inner muscle or tissue layer “M” (e.g., anterior rectus sheath) that enshroud organs, vessels, and/or other tissue for performing various bodily functions such as digestion. After the size of the bowel “B” is measured, the cannula 54 and the bowel measuring instrument 50 can be removed from the incision “I”. Although not described in detail herein, the bowel measuring device 50 can be as described in U.S. Application Serial No. (203-11699) which is incorporated herein by reference in its entirety.

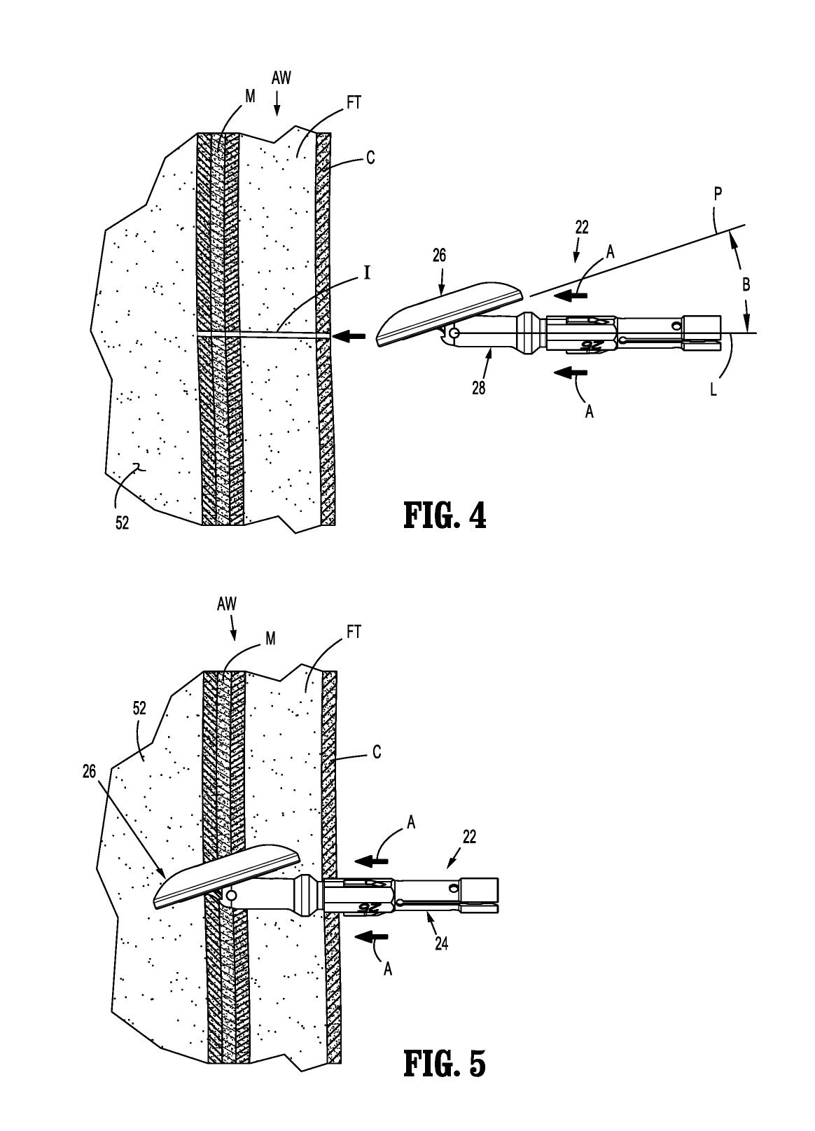

Referring to FIGS. 4-6, after the diameter of the bowel “B” to be exteriorized is measured, this measurement can be used to identify the size circular stapling device 10 (FIG. 1) best suited for the particular surgical procedure. As discussed above, the appropriate size circular stapling device 10 can be selected by choosing a circular stapling device that forms a hole “O” (FIG. 10) in the abdominal wall “AW” that corresponds in size to the outer diameter of the portion of the bowel “B” to be exteriorized.

Once the appropriate sized circular stapler 10 (FIG. 1) is selected, the anvil assembly 22 of the circular stapling device 10 is inserted through the incision “I”. As shown, the anvil assembly 22 should be inserted through the incision “I” (FIG. 4) in the direction indicated by arrows “A” in FIGS. 4 and 5 with the anvil head assembly 26 in a tilted position. In the tilted position, a plane “P” (FIG. 4) defined by a tissue contact surface 26a of the anvil head assembly 26 defines an acute angle β with a longitudinal axis “L” of the center rod assembly 24 such that the anvil head assembly 26 defines a reduced profile. This reduced profile of the anvil head assembly 26 minimizes trauma to a patient during insertion and removal of the anvil assembly 22 into/from a body cavity, e.g., the abdominal cavity 52 and minimizes the size of the incision “I” required to introduce the anvil head assembly 26 into the abdominal cavity 52. The anvil head assembly 26 should be advanced in the direction of arrow “A” until the anvil head assembly 26 is fully positioned within the abdominal cavity 52. In this position, the anvil head assembly 26 is positioned internally of the inner muscle “M” of the abdominal wall “AW”.

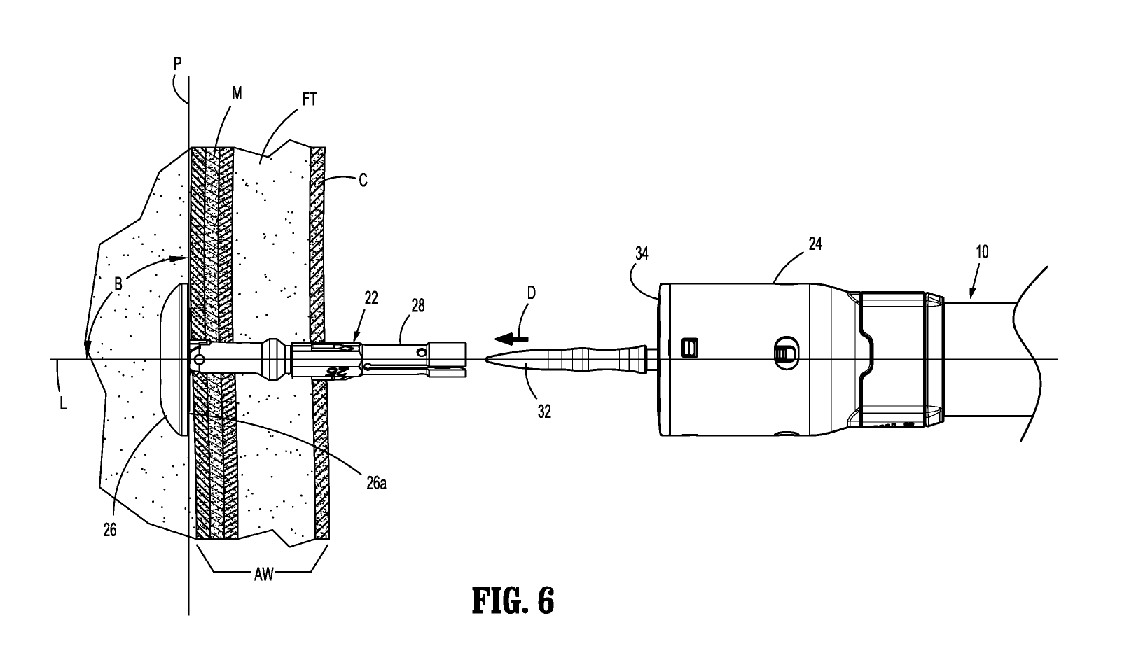

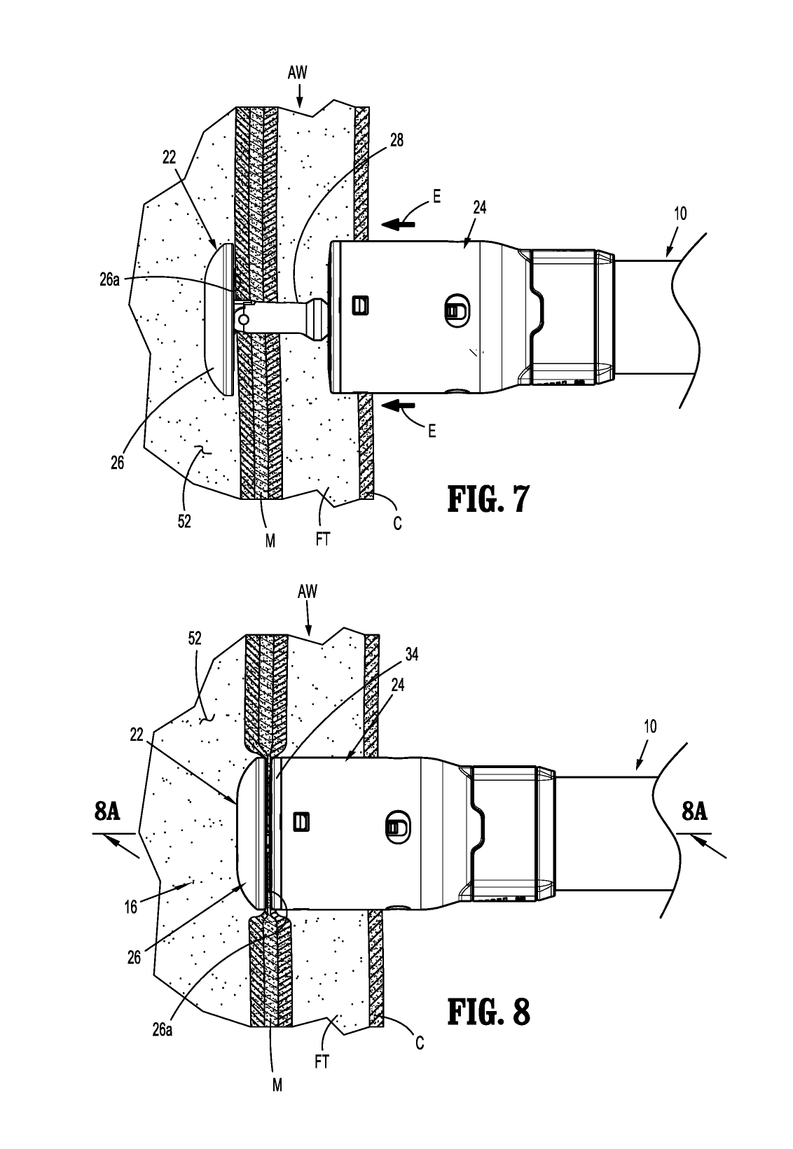

Referring to FIGS. 6 and 7, once the anvil head assembly 26 of the anvil assembly 22 is positioned within the abdominal cavity 52, the anvil head assembly 26 can be moved from the tilted position to the operative position. In the operative position, the plane “P” (FIG. 6) of the tissue contact surface 26a of the anvil head assembly 26 is substantially perpendicular to the longitudinal axis “L” of the center rod assembly 28. The anvil retainer 32 of the circular stapling device 10 (FIG. 1) can now be inserted into the center rod assembly 28 of the anvil assembly 22 in the direction indicated by arrow “D” in FIG. 6 to secure the anvil assembly 22 to the circular stapling device 10. It is envisioned that in some embodiments of the presently described method, the anvil assembly 22 can be supported on the anvil retainer 32 prior to insertion of the anvil assembly 22 through the incision “I”. Thereafter, the shell assembly 24 can be advanced through the incision “I” such that the inner muscle “M” of the abdominal wall “AW” is positioned between the staple cartridge 34 of the shell assembly 24 and the anvil head assembly 26 of the anvil assembly 22.

Referring to FIGS. 7 and 8, after the anvil assembly 22 is positioned within the abdominal cavity 52 and attached to the anvil retainer 32 (FIG. 6) of the circular stapling device 10 and the shell assembly 24 is passed through the incision “I” in the direction indicated by arrows “E” in FIG. 7, the anvil assembly 22 can be moved towards the shell assembly 24 to move the staple cartridge 34 and the anvil head assembly 26 to the clamped position to approximate the tool assembly 16 and clamp the inner muscle “M” of the abdominal wall (e.g., anterior rectus sheath) between the staple cartridge 34 of the shell assembly 24 and the tissue contact surface 26a of the anvil head assembly 26 of the anvil assembly 22.

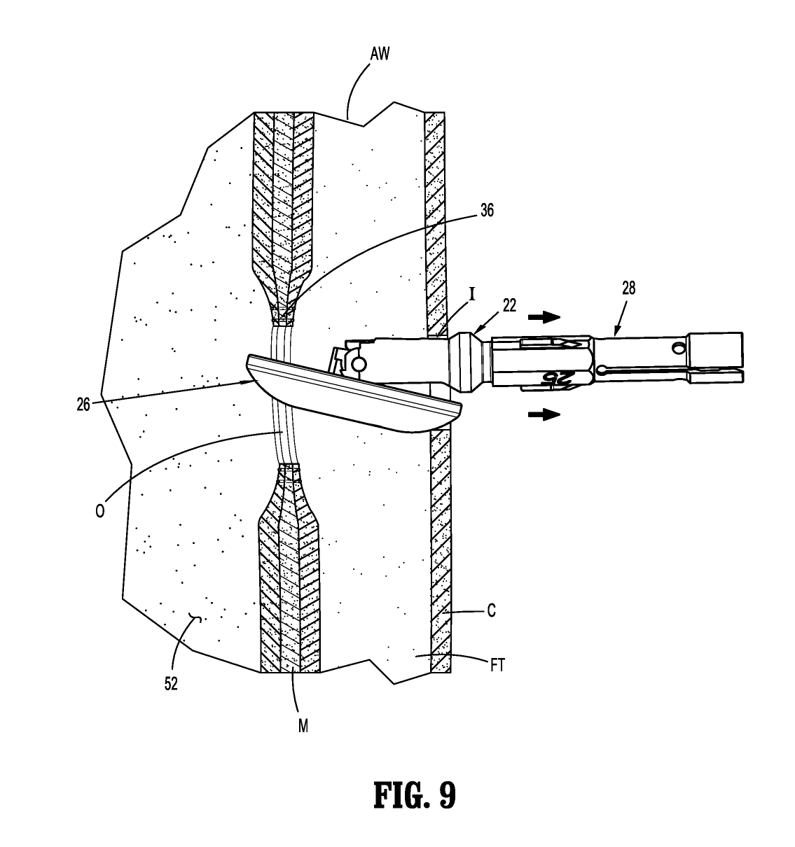

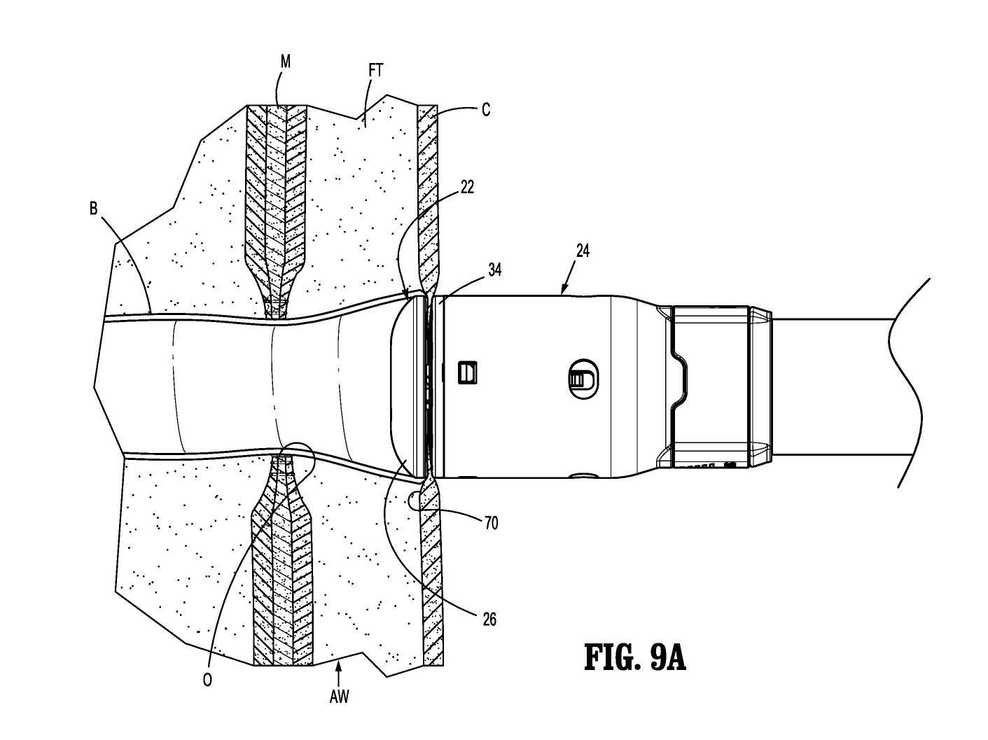

Referring to FIGS. 8 and 8A, after the inner muscle “M” is properly positioned between the staple cartridge 34 of the shell assembly 24 and the tissue contact surface 26a of the anvil head assembly 26, the circular stapling device 10 is fired by compressing the firing trigger 20 (FIG. 1). The shell assembly 24 includes an annular knife 60 and a pusher 62 (FIG. 8A). When the circular stapling device 10 is fired, the annular knife 60 and the pusher 62 are advanced towards the anvil head assembly 26 of the anvil assembly 22 to form a circular opening “O” (FIG. 9) in the inner muscle “M” and provide a circular array of the staples 36 about the opening “O” to reinforce the opening “O”.

Referring to FIG. 9, after the opening “O” is formed in the muscle layer “M”, the anvil assembly 22 can be unclamped using the rotation knob 18 and subsequently separated from the anvil retainer 20 of the circular stapling device 10. The anvil head assembly 26 can be returned to a tilted position and removed from the abdominal cavity 52 through the incision “I”. Alternately the circular stapling device 10 can be removed from the incision “I” with the anvil assembly 22 attached to the anvil retainer 32 of the circular stapling device 10 in the unclamped tilted position.

After the circular stapling device 10 including the anvil assembly 16 is removed from the patient through the incision “I, the shell assembly 24 can be replaced with a fresh shell assembly 24 having an unfired staple cartridge 34. See, e.g., the '590 Application for a detailed description of replacement of the shell assembly.

Referring to FIGS. 9A-11, after a fresh shell assembly 24 is secured to the body 14 of the circular stapling device, the bowel “B” can be pulled through the opening “O” in the inner muscle “M” of the abdominal wall “AW” and positioned adjacent the dermis 70 of the outer cutaneous layer “c”. Thereafter, the anvil head assembly 26 can be repositioned through the incision “I” (FIG. 9) and approximated with the staple cartridge 34 of the shell assembly 24 to clamp an outer end of the bowel “B” against the dermis 70 of the outer cutaneous layer “c” of the abdominal wall “AW”. After the outer end of the bowel “B” is properly clamped against the dermis 70 of the outer cutaneous layer “c” of the abdominal wall “AW”, the circular stapling device 10 can be fired as discussed above to secure the bowel “B” to the abdominal wall “AW” with an annular array of staples 36 to form the stoma “ST” (FIG. 10). At this point, the circular stapling device 10 can be unclamped to allow the anvil head assembly 26 to tilt and the anvil assembly 26 can be removed from the patient through the incision “I”.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A method of performing an ostomy procedure comprising:
forming an incision in an abdominal wall of a patient;
inserting an anvil head assembly of an anvil assembly through the incision with the anvil head assembly in a tilted position in relation to a center rod assembly of the anvil assembly;
positioning the anvil head assembly in the abdominal cavity of the patient;
moving the anvil head assembly from the tilted positon to an operative position;
coupling the anvil assembly to an anvil retainer of the circular stapling device;
inserting a shell assembly of the circular stapling device through the incision;
approximating the anvil head assembly in relation to a staple cartridge of the shell assembly to clamp the rectus sheath of the abdominal wall between the anvil head assembly and the staple cartridge; and
firing the circular stapling device to form a circular opening in the rectus sheath that is reinforced by an annular array of staples.

2. The method of claim 1, further including removing the anvil assembly from the abdominal cavity.

3. The method of claim 2, wherein removing the anvil assembly from the abdominal cavity is conducted through the incision.

4. The method of claim 3, further including moving the anvil head assembly to the tilted position prior to removing the anvil head assembly from the abdominal cavity.

5. The method of claim 4, further including separating the anvil assembly from the anvil retainer before removing the anvil assembly from the abdominal cavity.

6. The method of claim 1, further including securing a portion of a bowel of a patient to the abdominal wall to form a stoma.

7. The method of claim 6, wherein securing a portion of the bowel to the abdominal wall includes securing the portion of the bowel to a dermis of the abdominal wall of the patient.

8. The method of claim 7, wherein securing the portion of the bowel of the patient to the abdominal wall is performed with the circular stapling device.

9. The method of claim 8, wherein securing the portion of the bowel of the patient to the abdominal wall includes inserting the anvil head assembly of the circular stapling device through the incision and clamping the portion of the bowel to the outer cutaneous layer of the abdominal wall to form a stoma.

10. The method of claim 9, forming a circular opening in the outer cutaneous layer of the abdominal wall.

11. The method of claim 10, further including removing the anvil assembly from the stoma.

12. The method of claim 1, further including measuring a diameter of a section of bowel to determine an appropriate size for the circular stapling device.
